(19) United States
(12) Patent Application Publication
(10) Pub. No.: US 2022/0372471 A1
(43) Pub. Date: Nov. 24, 2022

(54) APPARATUSES SYSTEMS AND METHODS FOR ENRICHMENT AND SEPARATION OF NUCLEIC ACIDS BY SIZE

(71) Applicant: Apostle, Inc., Menlo Park, CA (US)

(72) Inventors: Dongliang Ge, Redwood City, CA (US); Shuting Zhao, Santa Clara, CA (US); Hao Wan, Palo Alto, CA (US); Bo Zhang, San Carlos, CA (US); Xin Guo, Palo Alto, CA (US); Wenqi Zeng, Irvine, CA (US)

(73) Assignee: Apostle, Inc., Menlo Park, CA (US)

(21) Appl. No.: 17/761,389

(22) PCT Filed: Jul. 10, 2020
(86) PCT No.: PCT/US2020/041686

§ 371 Date: Mar. 17, 2022

Related U.S. Application Data
(60) Provisional application No. 62/902,155, filed on Sep. 18, 2019.
(60) Provisional application No. 62/932,229, filed on Nov. 7, 2019.

(51) Int. Cl. C12N 15/10; C12Q 1/6869

(52) U.S. Cl. CPC C12N 15/1093; C12N 15/1013; C12Q 1/6869

(57) ABSTRACT

Embodiments of the disclosure are drawn to apparatuses, systems, and methods for enrichment and separation of nucleic acids by size. A sample may include a mixture of nucleic acids of various sizes, and the nucleic acids of interest may be below a particular size threshold. An example enrichment method may include mixing the sample with a first substrate (e.g., magnetic beads). The method may include separating nucleic acids above a first size threshold form a remainder of the sample using the first substrate. The method may include mixing the nucleic acids in the remainder of the sample (e.g., nucleic acids below’ the size threshold) with a second substrate and recovering the nucleic acids below the first size threshold from the second substrate.

### CROSS-REFERENCE TO RELATED APPLICATION

This application claims the filing benefit of U.S. Provisional Application No. 62/902,155, filed Sep. 18, 2019 and U.S. Provisional Application No. 62/932,229, filed Nov. 7, 2019. These applications are incorporated by reference herein in their entireties and for all purposes.

### BACKGROUND

There may be many applications where it is useful to separate target biomolecules from a solution or to enrich the population of target biomolecules within a solution. Many biological solutions (e.g., patient samples, laboratory mixtures, etc.) may include a mix of target biomolecules along with other non-target components (non-targeted biomolecules, salts, buffer components, cells, etc.). Different procedures have been developed with the goal of extracting and/or enriching the target biomolecules, for example to remove undesirable components of the original solution and/or to increase the proportion of the target products compared to non-target products. It may be desirable to separate target biomolecules from other components of the solution (such as non-targeted biomolecules) based on the size of the target biomolecules.

### SUMMARY

In at least one aspect, the present disclosure relates to a method. The method includes mixing a sample including nucleic acids with a first substrate. The method includes separating nucleic acids above a first size threshold from a remainder of the sample with the first substrate. The method includes mixing the nucleic acids in the remainder of the sample with a second substrate. The method includes recovering nucleic acids below the first size threshold from the second substrate

The method may also include recovering the nucleic acids above the first size threshold. The method may also include binding the nucleic acids above the first size threshold to a first substrate including magnetic beads, and recovering the nucleic acids above the first size threshold may include eluting the nucleic acids above the first size threshold from the magnetic beads.

The first substrate may include a first population of magnetic beads, and the second substrate may include a second population of magnetic beads. The first population of magnetic beads and the second population of magnetic beads may include core-shell-shell magnetic beads. Separating the bound nucleic acids above the first size threshold may include applying a magnetic field to the sample and removing a supernatant which includes the remainder of the sample. The first population of magnetic beads may include surface chemistry of carboxylic groups which may selectively bind to the nucleic acids above the first size threshold. The second population of magnetic beads may include surface chemistry of hydroxyl groups. The method may also include binding the nucleic acids in the remainder of the sample to the second population of magnetic beads by mixing the remainder of the sample with a nucleic acid precipitation reagent including at least one alcohol.

The method may also include separating nucleic acids above a second size threshold from the remainder of the sample with the second substrate. The second size threshold may be smaller than the first size threshold, and the recovered nucleic acids may be above the second size threshold and below the first size threshold.

The method may also include binding the nucleic acids above the first size threshold to the first substrate in the presence of a first nucleic acid precipitation reagent and binding the nucleic acids in the remainder of the sample to the second substrate in the presence of a second nucleic acid precipitation reagent. At least one of the first nucleic acid precipitation reagent and the second nucleic acid precipitation reagent may include dehydrating agents, salt bridges, buffering agents, carrier molecules, surfactant, and combinations thereof.

The method may also include washing the second substrate with a wash buffer. Recovering the nucleic acids below the first size threshold from the second substrate may include eluting the nucleic acids from the second substrate with an elution buffer. The nucleic acid may include DNA, RNA, oligos, nucleic acids labeled with radioactive phosphates, fluorophores, nucleotides modified with biotin or digoxygenin, or combinations thereof. The first size threshold may be 100 bp, 110 bp, 120 bp, 130 bp, 140 bp, 150 bp, 160 bp, 170 bp, 180 bp, 190 bp, or 200 bp. The first substrate may include magnetic beads, non-magnetic beads, a gel, a capillary tube, or a spin column and the second substrate may include magnetic beads, non-magnetic beads, a gel, a capillary tube, or a spin column. Separating the nucleic acids above the first size threshold may include applying a magnetic field, applying an acceleration, centrifuging the sample, or applying a potential to the first substrate.

In at least one aspect, the present disclosure relates to a kit which includes a first substrate, a first nucleic acid precipitation reagent, a second substrate, and a second nucleic acid precipitation reagent. The first substrate may bind nucleic acids above a first size threshold in the presence of the first nucleic acid precipitation reagent, and the second substrate may bind nucleic acids in the present of the second nucleic acid precipitation reagent.

The first substrate may be a first population of magnetic beads, and the second substrate may be a second population of magnetic beads. The first population of magnetic beads and the second population of magnetic beads may include core-shell-shell magnetic beads. The second substrate may bind nucleic acids above a second size threshold in the presence of the second nucleic acid precipitation reagent.

At least one of the first nucleic acid precipitation reagent or the second nucleic acid precipitation reagent may include dehydrating agents, salt bridges, buffering agents, carrier molecules, surfactant, and combinations thereof. The dehydrating agents may include polyalkylene glycols with a molecular weight between 1000 to 10,000, and a weight to volume concentration between 10% to 25%. The salt bridge may include NaCl, KCl, CaCl₂) or combinations thereof, with a concentration from 0.5M to 5M. The buffering agents may include Tris with a concentration of 0-10 mM, EDTA with a concentration of 0-1 mM, Tris-HCl with a concentration of 0-10 mM, and combinations thereof. The carrier molecules may include Sodium Acetate with a concentration of 0.3-1M, Lithium Chloride with a concentration of 0.1-1M, Glycogen with a concentration of 0.1-2 uM, Ammonium acetate with a concentration of 0.5-2M, Linear Polyacrylamide with a concentration of 10-20 ug/mL, and combinations thereof. The surfactant may include Tween-20 with a concentration of 0.01%-0.5%, Triton X-100 with a concentration of 0.01%-0.5%, SDS with a concentration of 0.1%-1%, and combinations thereof.

The kit may also include an alcohol comprising ethanol with a concentration of 45-85%, isopropanol with a concentration of 33%-70%, and combinations thereof. The kit may, also include a washing buffer including at least one alcohol. The kit may also include an elution buffer including Tris with a concentration of 0-10 mM, EDTA with a concentration of 0-1 mM, Tris-HCl with a concentration of 0-10 mM, and combinations thereof.

In at least one aspect, the present disclosure may relate to a method. The method includes collecting a sample including nucleic acids. The method includes isolating the nucleic acids from the sample. The method includes filtering the nucleic acids to retain selected ones of the nucleic acids below a size threshold using a first substrate which may selectively separate nucleic acids above the size threshold from the selected ones of the nucleic acids and a second substrate which may separate the selected ones of the nucleic acids from impurities. The method includes preparing a library based on the isolated nucleic acids. The method includes sequencing the prepared library.

The filtering may occur after isolating the nucleic acids and before preparing the library. Preparing the library may include elongating the isolated nucleic acids. Filtering the nucleic acids may occur after the isolated nucleic acids are elongated. The size threshold may be based on a target length of nucleic acids and an amount of elongation.

The nucleic acids may be cell free DNA (cfDNA), DNA, RNA, oligos, labelled nucleic acids, modified nucleic acids, or combinations thereof. The nucleic acids below the size threshold may include fetal DNA. The nucleic acids below the size threshold may include tumor DNA.

The method may also include recovering the nucleic acids above the size threshold. Filtering the nucleic acids may include binding the non-selected ones of the nucleic acids to the first substrate and applying an external force to separate the first substrate and the bound non-selected ones of the nucleic acids from unbound ones of the nucleic acids. The first substrate may include magnetic beads and the external force may be a magnetic field. The filtering may also include binding the selected ones of the nucleic acids to the second substrate, applying an external force to separate the second substrate and the bound selected ones of the nucleic acids from the impurities, and recovering the selected ones of the nucleic acids from the second substrate.

### BRIEF DESCRIPTION OF THE DRAWINGS

FIG. 1 is a flow chart of a method of filtering nucleic acids based on size according to some embodiments of the present disclosure.

FIG. 2 is a schematic diagram of a method of enriching and purifying short DNA fragments according to some embodiments of the present disclosure.

FIG. 3 is a cross-sectional diagram of a core-shell-shell magnetic bead according to some embodiments of the present disclosure.

FIGS. 4A-4B are graphs depicting the separation of nucleic acids by size according to some embodiments of the present disclosure.

FIGS. 5A-5C are graphs depicting enrichment of small DNA fragments from a cell-free DNA (cfDNA) sample according to some embodiments of the present disclosure.

FIGS. 6A-6C are graphs of enrichment of small DNA fragments from human cell-free DNA (cfDNA) sample according to some embodiments of the present disclosure.

FIGS. 7A-7D are graphs showing the enrichment of a fetal fraction of DNA assessed by the quantification of the Y chromosome according to some embodiments of the present disclosure.

### DETAILED DESCRIPTION

The following description of certain embodiments is merely exemplary in nature and is in no way intended to limit the scope of the disclosure or its applications or uses. In the following detailed description of embodiments of the present systems and methods, reference is made to the accompanying drawings which form a part hereof, and which are shown by way of illustration specific embodiments in which the described systems and methods may be practiced. These embodiments are described in sufficient detail to enable those skilled in the art to practice presently disclosed systems and methods, and it is to be understood that other embodiments may be utilized and that structural and logical changes may be made without departing from the spirit and scope of the disclosure. Moreover, for the purpose of clarity, detailed descriptions of certain features will not be discussed when they would be apparent to those with skill in the art so as not to obscure the description of embodiments of the disclosure. The following detailed description is therefore not to be taken in a limiting sense, and the scope of the disclosure is defined only by the appended claims.

An increasing number of biological assays are based on the nucleic acids in a sample (e.g., a biological fluid, the output of a laboratory assay, amplicons, etc.). A sample may include a mixture of nucleic acids of interest along with other nucleic acids. In some applications, the nucleic acids of interest may be differentiated from the other nucleic acids based on their size. Accordingly, it may be useful to process a sample in order to separate the nucleic acids based on their size in order to separate target nucleic acids from the other nucleic acids. In some example applications, the target nucleic acids may generally be those which are below a size cutoff.

One such example application is the enrichment and separation of short nucleic acid fragments (e.g. less than 150 bp DNA) to distinguish or enrich fetal DNA, tumor-derived cell free DNA (cfDNA), or other short nucleic acids of interest from background nucleic acids of various size. This may be useful to improve the sensitivity of subsequent molecular biological process technologies and diagnostic platforms. For example, in non-invasive prenatal testing (NIPT) at least 4% fetal fraction of cfDNA may be desirable for accurate diagnosis of common chromosomal abnormalities and an even higher fetal fraction may be desirable for the detection of monogenic diseases. A sample from a mother (e.g., a blood draw) may include a mixture of fetus-derived cell-free DNA (cffDNA) and maternal cfDNA, however the cffDNA may generally be shorter than the maternal cfDNA in the sample. By increasing the short DNA fragment fraction (e.g., by separating cffDNA from maternal DNA and/or selectively enriching the concentration of cffDNA), the fetal fraction could potentially increase, which may benefit the NIPT accuracy.

In another example application, enrichment and/or separation of nucleic acids by size may aid in the detection and/or identification of tumors (e.g., cancer diagnosis). Tumor-derived DNA molecules or cell-free tumor DNA (cfDNA) may have altered fragmentation profiles and the ratio of short (100-150 bp) to long (151-220 bp) cfDNA of cancer patients may be useful to identify cancers and tissue of origin for liquid biopsy purpose. In some applications, the detection/identification of disease in a patient may be possible. For example, short nucleic acids localized in exosomes or small DNA fragments in urine may be used as indicators for diseases diagnosis. Increased percentage of nucleic acids of interest may reduce background noise and facilitate the depth of sequencing for genome-wide and mutational map. There may thus be a need for assay platforms which allow for size selective enrichment/separation of nucleic acids in a sample.

The present disclosure is drawn to apparatuses, systems, and methods for enrichment and separation of nucleic acids by size. An example method may include mixing a sample (e.g., a patient sample such as a biological fluid, cultured cells, the product of a lab assay such as PCR, etc.) which includes nucleic acids of various sizes with a first substrate. Nucleic acids above a first size threshold may then be separated from a remainder of the sample using the first substrate. The remainder of the sample, which includes nucleic acids below the first size threshold may then be mixed with a second substrate. The second substrate may allow for the separation of impurities from the filtered short nucleic acids (e.g., by washing) and/or enrichment of the sample by concentrating the short nucleic acids. The nucleic acids below the first size threshold may then be recovered. For example, if the nucleic acids were bound to the second substrate, they may be eluted, and separated from the second substrate. In some embodiments, the second substrate may also be size selective for nucleic acids above a second size threshold, and the recovered nucleic acids may be between the first and second size thresholds. The use of two substrates may allow for efficient size selective separation/enrichment, since the first substrate may be selected for its size selective properties (e.g., strong binding affinity for nucleic acids below the size threshold), while the second substrate may be selected for advantageous effects such as high binding affinity to all nucleic acids, a secondary size selection, etc.

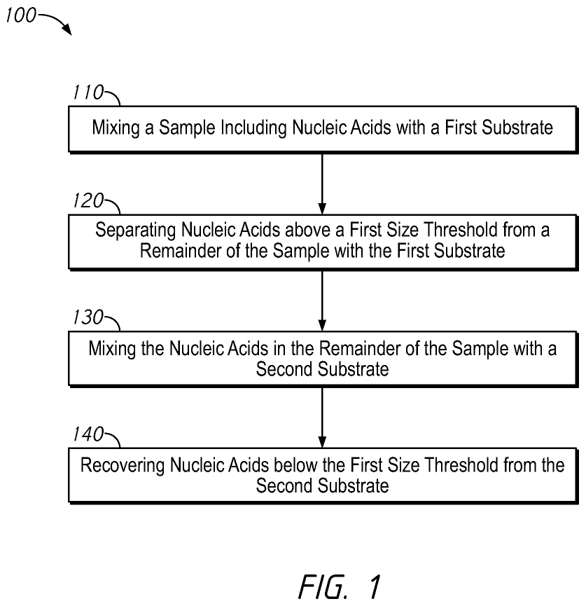

FIG. 1 is a flow chart of a method of filtering nucleic acids based on size according to some embodiments of the present disclosure.

The method 100 may generally begin with block 110, which describes mixing a sample including nucleic acids with a first substrate. The sample may be any solution or mixture which includes nucleic acids. In some embodiments, the nucleic acids may include DNA, RNA, oligos, labelled nucleic acids such as nucleic acids labeled with radioactive phosphates and/or fluorophores, modified nucleic acids, such as nucleic acids including nucleotides modified with biotin or digoxygenin, or combinations thereof. In some embodiments, the sample may be a biological sample, such as a fluid obtained from a patient, for example, blood, tissues, serum, plasma, lymph, urine, semen, saliva, milk, cultured cells and combinations thereof. In some embodiments, a biological sample may be obtained from a non-human subject, such as a plant or animal, or may be from an artificial source, such as a cell line cultured in a laboratory. In some embodiments, the sample may the product of a previous reaction or assay. For example, the sample may be the product of a molecular biology assay such as gene sequencing, an amplification reaction (e.g., qPCR, ddPCR, isothermal amplification, etc.), and/or other processes.

In some embodiments, before the block 110, the method 100 may additionally include preparing the sample. For example, if the sample is a biological fluid which includes cells, the preparation may include lysing cells and/or filtering cells (or cellular debris) out of the sample. Another example of preparing the sample may be performing an amplification reaction to increase a copy number of nucleic acids in a sample.

Block 110 may generally be followed by block 120, which describes separating nucleic acids above a first size threshold from a remainder of the sample with the first substrate. The first substrate may be a substrate which is capable of separating the nucleic acids by size. In some examples, the first substrate may separate nucleic acids above a size threshold from those which are below a size threshold. The first substrate may intrinsically separate nucleic acids by size and/or may selectively filter by size in the presence of one or more other reagents, external forces, etc. The details of block 120 (as well as the mixing described in block 110) may depend on the type of substrate used as the first substrate. Some example substrates and separation methods are given below.

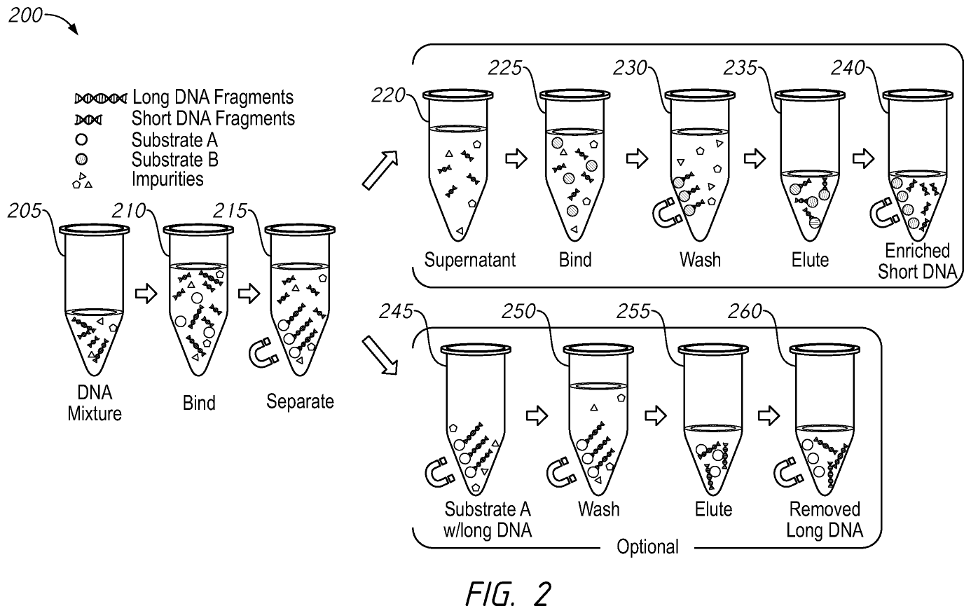

In some embodiments, the first substrate may be a population of magnetic beads, which may bind to nucleic acids in the presence of a nucleic acid precipitation reagent. The surface chemistry and the nucleic acid precipitation reagent may cause nucleic acids above the size cutoff to preferentially bind to the magnetic beads. The type of surface chemistry and nucleic acid precipitation reagent may determine the size cutoff. In some embodiments, the size cutoff may be tunable based on the chosen surface chemistry and nucleic acid precipitation reagent. An example embodiment using magnetic beads is described in more detail in FIG. 2.

In some embodiments where the first substrate includes magnetic beads, block 110 may include mixing the sample with the magnetic beads and the nucleic acid precipitation reagent, and binding nucleic acids above the first size threshold to the magnetic beads in the presence of the nucleic acid precipitation reagent. Block 120 may include applying a magnetic field to the sample to separate the magnetic beads (and the bound nucleic acids above the size threshold) from a liquid phase of the sample (which may include the unbound nucleic acids below the size threshold). The supernatant may then be removed and transferred to another container.

In some embodiments, the first substrate may be a population of beads and centrifugation may be used to separate the beads (and the nucleic acids they are bound to). The beads may be magnetic or non-magnetic. Block 110 for beads with centrifugation may generally be similar to block 110 as described for magnetic beads (e.g., binding nucleic acids above a size threshold to the beads based on surface chemistry of the beads and a precipitation reagent). Block 120 may include applying an acceleration to the sample to separate the beads and the nucleic acids bound to the beads from the rest of the sample. For example, the sample may be centrifuged to pull the beads (and bound nucleic acids) out of the solution, so that the supernatant (including the shorter nucleic acids) may be removed.

In some embodiments, the first substrate may be a gel, which may be used for electrophoresis. Block 110 may include loading the sample onto the gel. Block 120 may, include applying a potential to the gel, (e.g., by applying a voltage differential across the gel). The nucleic acids in the sample may migrate through the gel at a rate proportional to their size. A size standard (e.g., a ladder) may be run to provide comparison for how far nucleic acids of different size have migrated through the gel. Block 120 may also involve removing the nucleic acids below the size threshold from the gel, for example by cutting out a portion of the gel containing those nucleic acids, and then releasing the nucleic acids from the gel. Other types of electrophoresis, such as capillary tube electrophoresis, may also be used in other embodiments.

In some embodiments, the first substrate may be a column-based separation medium. For example, a spin column such as a silica membrane column may be used. The sample may be loaded into the spin column, and then an acceleration may be applied to the column (e.g., with centrifugation) to separate the nucleic acids by size.

It should be understood that the method 100 may also include additional steps which are dependent on the type of substrate chosen for the first substrate. For example, if gel electrophoresis is used, the method 100 may include steps such as pouring the gel and preparing a running buffer.

During the separation of block 120, the population of nucleic acids in the sample is separated into a first portion of the population of nucleic acids which is generally below the size cutoff, and a second portion of the population of nucleic acids which is above the size cutoff. Since the size of nucleic acids is generally characterized by a number of base pairs (bp) or a number of nucleotides (nt), the size cutoff may be represented by a number of base pairs or nucleotides. The value of the size cutoff may depend on the type of nucleic acids of interest. In some embodiments, the size cutoff may be inherent to the type of substrate chosen as the first substrate. In some embodiments, the size cutoff may be tunable and may be chosen based on the reaction conditions used with the first substrate (e.g., the type of nucleic acid loading buffer). In some embodiments, the substrate may separate nucleic acids by size, and a user may choose the size cutoff based on which portions of the separated nucleic acids they select (e.g., by cutting out certain bands of a gel).

In some embodiments, an example size cutoff may be 150 bp. Larger or smaller size cutoffs may be used in other embodiments. For example, the size cutoff may be 100 bp, 110 bp, 120 bp, 130 bp, 140 bp, 150 bp, 160 bp, 170 bp, 180 bp, 190 bp, 200 bp, 210 bp, 220 bp, 230 bp, 240 bp, 250 bp, 260 bp, 270 bp, 280 bp, 290 bp, 300 bp, 310 bp, or 320 bp. In some examples, cutoff sizes smaller than 100 bp or larger than 320 bp may be used. In some embodiments the cutoff size may represent a size difference in the nucleic acids in their native condition (e.g., fetal vs. maternal cfDNA). In some embodiments, the method 100 may include an additional step of fractioning the nucleic acids. This may cause relatively long nucleic sequences (e.g., genomic DNA) to be sliced into a number of relatively small pieces (e.g., fragments). The size cutoff may represent a cutoff between longer fragments and shorter fragments. The amount of shorter fragments and/or the ratio of shorter to longer fragments may be a useful measurement in certain applications (e.g., tumors may have a different ratio than non-tumors).

Block 120 may generally be followed by block 130, which describes mixing the nucleic acids in the remainder of the sample with a second substrate. After blocks 110-120, the remainder of the sample may generally contain nucleic acids which are below the size cutoff. However, the remaining sample may also be filled with various impurities, which may be leftover from the first substrate and/or the original sample. Blocks 130 (and 140) describe steps which may be used to purify and/or enrich the size filtered sample. In addition to enriching/purifying the size filtered sample, blocks 130-140 may be used to apply additional size selective filtration.

Block 130 may include the use of a second substrate. In some embodiments, the second substrate may be the same type of substrate used as the first type of substrate. In some embodiments, the second substrate may be a different type of substrate than the type of substrate used as the first type of substrate. The second substrate may have a different size threshold (or no size threshold) than the first size threshold applied by the first substrate in blocks 110-120. In embodiments where the second substrate is the same type of substrate as the first substrate, then different conditions may be used to change the size cutoff. For example, if magnetic beads are used as both the first and second substrate, then different bead surface chemistry and/or nucleic acid precipitation reagents may be used to control the size cutoff (or lack thereof). Other reaction conditions (e.g., physical conditions such as temperature or timing) may also be used to modify the behavior of different substrates of the same type.

Block 130 may generally be followed by block 140, which describes recovering nucleic acids below the first size threshold from the second substrate. Blocks 130-140 may be generally similar to the blocks 110 to 120, and may be based on the type of substrate chosen as the second substrate.

In some embodiments, the second substrate may be used to separate nucleic acids above a second size threshold from nucleic acids below the first size threshold. The second size threshold may be smaller than the first size threshold. Accordingly, the recovered nucleic acid in block 140 may be above the second size threshold and below the first size threshold.

In some embodiments, block 140 may include enriching the nucleic acids below the first size threshold by increasing a concentration of the nucleic acid below the first size threshold. For example, blocks 130-140 may include separating the nucleic acids below the first size threshold from a liquid phase and then resuspending the nucleic acids in a smaller volume of fluid.

The use of both a first substrate and second substrate together in the method 100 may lead to a synergistic effect which allows for the efficient recovery and/or enrichment of relatively small nucleic acid fragments. For example, a substrate selected as the first substrate may be able to efficiently separate nucleic acids by size (e.g., have strong size selectivity), but may not be efficient at the recovery of the smaller nucleic acids. Similarly, a substrate selected as the second substrate may be efficient at recovering nucleic acids, but may demonstrate poor size selective capabilities. By using both substrates as part of the method 100, a strong size selective filtration may be achieved along with the efficient recovery of the nucleic acids below the size threshold. In addition, by using two different substrates, each may be separately ‘tuned’ (e.g., based on the chemistry of the substrate, the chemistry of various buffers, various physical reaction conditions, etc.) to maximize the size selective separation and recovery respectively.

FIG. 2 is a schematic diagram of a method of enriching and purifying short DNA fragments according to some embodiments of the present disclosure. The method 200 may, in some embodiments, be an implementation of the method 100 of FIG. 1. The method 200 is a method which uses DNA as the nucleic acids, and magnetic beads as the first and second substrates (e.g., the first and second substrate of FIG. 1). It should be understood that methods similar to the method 200 may be used in which the first and/or second substrate are not magnetic beads, and in which the nucleic acids include nucleic acids other than DNA. The method 200 shows a number of blocks 205-260, represented as reaction tubes, each of which may represent a portion of the method 200. Although the method 200 is shown taking place in microcentrifuge tubes, it should be understood that this is for illustration purposes only, and that any type of container may be used to contain the steps of the reaction, and that in some embodiments the reaction may be transferred to different containers in different steps of the reaction.

The method 200 may include block 205, which shows a DNA mixture. The DNA mixture includes long DNA fragments (e.g., DNA above a size cutoff), short DNA fragments (e.g., DNA below the size cutoff), and impurities. The DNA mixture may be a patient sample which may have been treated (e.g., fragmented) by one or more processes. The DNA mixture may be the output of a previous reaction (e.g., an amplification reaction). While the sample of block 205 is shown as a mixture of DNA, it should be understood that mixtures of other types of nucleic acid may also be used, such as DNA, RNA, oligos, combinations thereof, etc.

Block 205 may generally be followed by block 210, which includes mixing a first substrate (e.g., a first population of magnetic beads) and a first DNA precipitation reagent with the DNA mixture (e.g., as in block 110 of FIG. 1). The block 210 may also include binding the long DNA fragments to the first population of magnetic beads. The long DNA fragments may specifically bind to the first population of magnetic beads based on the surface chemistry, of the magnetic beads and the composition of the DNA precipitation reagent.

The first population of magnetic beads may include surface chemistry, which in some embodiments can be functionalized, for example, with small molecules, polymers, and dendrimers with epitope carboxylic groups. The carboxylic groups on the surface of the first population of magnetic beads may bind the long DNA fragments (but not the short DNA fragments) in the presence of the first DNA precipitation reagent. The beads and nucleic acid precipitation reagent may bind the long nucleic acids fragments based on the mechanism of solid-phase reversible immobilization.

The nucleic acid precipitation reagent may include dehydrating agents, a salt bridge, buffering agents and combinations thereof. The dehydrating agents may provide a hydrophobic solution to force hydrophilic nucleic acid molecules out of solution, which in turn may facilitate the precipitation of long nucleic acids onto the magnetic beads. The dehydrating agents may include polyalkylene glycols (e.g., polyethylene glycol, polypropylene glycol, etc.) which in some embodiments may have a molecular weight of between 1000 to 10,000 Da, and a weight to volume concentration of between 10% to 25%.

The salt bridge may help minimize the negative charge repulsion of the long nucleic acid molecules. The salt bridge may include salts such as NaCl, KCl, CaCl₂), and combinations thereof. In some embodiments, the salts) of the salt bridge may have a concentration of between 0.5M to 5M.

The buffering agents may provide appropriate pH for long nucleic acids binding on the magnetic beads surface. The buffering agents may include Tris, Tris-HCl, EDTA, and combinations thereof. One example set of buffering agents included in a nucleic acid precipitation reagent may include Tris with a concentration of 0-10 mM, EDTA with a concentration of 0-1 mM, Tris-HCl with a concentration of 0-10 inn and combinations thereof.

The nucleic acid precipitation reagent may also include additional components. In some embodiments, a surfactant is added to the nucleic acids precipitation reagent to reduce non-specific nucleic acid binding. For example, surfactants such as Tween-20, Triton X-100, SDS, and combinations thereof may be used. One example set of surfactants may include Tween-20 with a concentration of 0.01%-0.5%, Triton X-100 with a concentration of 0.01%-0.5%, SUS with a concentration of 0.1%-1%, and combinations thereof.

In some embodiments, the nucleic acids precipitation reagent may be used to help determine the size cutoff. For example, increasing the strength (reagent concentration, volume to sample volume, etc.) of the nucleic acid precipitation reagent may decrease the cutoff size of the long versus short fragments.

Block 210 may generally be followed by block 215, which shows separating the long DNA fragments and first population of magnetic beads from a remainder of the sample (e.g., as in block 120 of FIG. 1). As part of block 215, a magnetic field may be applied to the sample. For example, an external magnet may be positioned near the container which holds the sample. Responsive to the application of the magnetic field, the first population of magnetic beads (and the long DNA fragments bound thereto) may be drawn to a region of the container closest to the magnet. This may cause the magnetic beads (and long DNA fragments) to form a pellet, separating the magnetic beads from a liquid supernatant of the sample. This may allow the supernatant to be removed from the container, separating the supernatant (including the short DNA fragments) from the pellet (including the long DNA fragments). In embodiments where magnetic beads are not used, a different method may be used as part of the block 215 to separate the nucleic acids, for example centrifugation may be used with non-magnetic beads or spin columns, and a voltage may be applied to an electrophoretic substrate.

Block 215 may generally be followed by block 220, which shows the supernatant. The supernatant may include the short DNA fragments and at least some of the impurities from the original sample, as well as one or more components of the first nucleic acid precipitation reagent. It should be understood that in some embodiments the supernatant may also include one or more of the first magnetic beads and long DNA fragments due to imperfect separation, however for the purposes of clarity, the illustration of FIG. 2 shows an example where substantially all of the first magnetic beads and long DNA fragments are removed.

Block 220 may generally be followed by block 225, which describes binding the short DNA fragments to a second population of magnetic beads in the presence of a second nucleic acid precipitation reagent (e.g., as in block 130 of FIG. 1). This may include mixing a second population of magnetic beads and a second nucleic acid precipitation reagent with the supernatant from block 220. In some embodiments, nucleic acids in the supernatant above a second size threshold may selectively bind to the second population of magnetic beads. In some embodiments, the binding of nucleic acids to the second population of magnetic beads may be non-size selective.

Similar to the first magnetic beads and first nucleic acid precipitation reagent, the lengths of nucleic acid bind which to the second population of magnetic beads may be based on the surface chemistry of the second population of magnetic beads and the composition of the second nucleic acid precipitation reagent. In some embodiments, the second population of magnetic beads and the second nucleic acid precipitation reagent may be non-size selective, and may bind all DNA fragments in the supernatant regardless of size.

In such embodiments, the second population of magnetic beads may include surface groups such as hydroxyl surface groups. The hydroxyl surface groups may bind to the short nucleic acids (e.g., by binding to nucleic acids in a manner non selective for size) in the presence of a second nucleic acid precipitation agent. The second nucleic acids precipitation reagent may include an alcohol, which facilitate the small fragments of nucleic acids to precipitate onto the hydroxyl groups on the surface of the second population of magnetic beads. In some embodiments, the alcohol may include ethanol, isopropanol or combinations thereof. In one example embodiment, the second nucleic acid precipitation reagent may include ethanol with a concentration of 45-85% and/or isopropanol with a combination of 33%-70%

In some embodiments, the second nucleic acid precipitation reagent may also include carrier molecules, which may enhance the precipitation of the nucleic acids. The carrier molecules may be soluble in aqueous solutions but may aggregate when the dielectric constant is lowered by the added alcohol. In some embodiments, the carrier molecules may include sodium acetate, lithium chloride, glycogen, ammonium, acetate, linear polyacrylamide, and combinations thereof. In some embodiments, the second nucleic acid precipitation reagent may include sodium acetate with a concentration of 0.3-1M, lithium chloride with a concentration of 0.1-1M, glycogen with a concentration of 0.1-2 uM, ammonium acetate with a concentration of 0.5-2M, linear polyacrylamide with a concentration of 10-20 ug/mL, and combinations thereof.

The second nucleic acid precipitation reagent may also include other components, such as components similar to the ones of the first nucleic acid precipitation reagent. In some embodiments, the second nucleic acid precipitation reagent may include carrier molecules, dehydrating agents, salt bridge, buffering agents, surfactants and combinations thereof. Similarly, in some embodiments the first nucleic acid precipitation reagent may include components, such as alcohols and carrier molecules, which were described with respect to the second nucleic acid precipitation reagent.

Additionally, and/or alternatively, the method 200 may include washing (at operation 230) following block 225. The washing may aid in the removal of components of the second (and/or first) nucleic acid precipitation reagent, along with remaining impurities. The washing block 230 may include applying a magnetic field (e.g., similar to block 215) to form a pellet and then removing the supernatant. The supernatant may then be replaced with a washing buffer. In some embodiments, the washing buffer may include at least one alcohol (e.g., similar to the alcohols in the second nucleic acid precipitation reagent). The washing block 230 may be repeated multiple times in some embodiments.

Block 230 may generally be followed by block 235, which describes eluting the short DNA fragments from the second population of magnetic beads. After a final wash block 230, the pellet may be resuspended in an elution buffer to the sample. The elution buffer may include components which cause the short DNA fragments to release from the magnetic beads and into the elution buffer. In some embodiments, the elution buffer may include a relatively low salt concentration to cause the release of the short DNA fragments. In some embodiments, the elution buffer may include a certain pH (e.g., pH from 6 to 10). In some embodiments, the elution block 235 may be used to concentrate (e.g., enrich) the short DNA fragments, by using a smaller volume of elution buffer than the volume of the original sample.

Block 235 may be followed by block 240, which shows the enriched short DNA fragments being separated from the second population of magnetic beads. This may include applying a magnetic field to form a pellet including the second population of magnetic beads, but not the eluted short DNA fragments which may generally remain in the supernatant. The supernatant including the short DNA fragments may then be removed for subsequent downstream assays. In some embodiments, the chemistry of the elution buffer may be chosen so that the elution buffer is compatible with downstream assays.

Additionally, the method 200 may also include blocks 245 to 260, which show the recovery of the long DNA fragments which were separated in block 215. Block 245 may include the pellet of the first population of magnetic beads and the long DNA fragments they are bound to. Block 245 may be followed by block 250, which describes washing the first magnetic beads and long DNA fragments. This may generally be similar to the wash block 230, and may include the use of a similar wash buffer. Block 250 may generally be followed by block 255 which includes eluting the long DNA fragments from the first population of magnetic beads. This may generally be similar to the elution block 235 and may involve the use of a similar elution buffer. Block 255 may generally be followed by block 260 which involves separating the eluted long DNA fragments from the second magnetic beads. Block 255 may generally be similar to the block 240. The recovered long DNA fragments may, in some embodiments, be useful in applications where it is desirable to compare a concentration of the long and short DNA fragments in the original sample. In some embodiments, the recovered long DNA fragments may be useful in various applications where purification/enrichment of the long fragments is preferred.

In some embodiments, the various reagents which are used in a particular implementation of the method 100 of FIG. 1 and/or 200 of FIG. 2 may be packaged together into a kit. In some embodiments, the kit may include chemical reagents, such as the first and second substrate, and reagents (e.g., the first and second nucleic acid precipitation reagents) used with those substrates. The kit may also include other components such as the wash buffer and elution buffer. In some embodiments, the kit may include physical components, such as a magnet and various (reusable and/or disposable) containers for mixing reagents. In some embodiments, the kit may include reagents pre-portioned for a single reaction. In some embodiments, the kit may include stocks of reagents which are measured out by the user for each individual reaction.

For example, the kit may include a mixture of reagents which may be used to perform the method 200 of FIG. 2. The kit may include the first population of magnetic beads, the second population of magnetic beads, the first nucleic acid precipitation reagent, and the second nucleic acid precipitation reagent. The first and second population of magnetic beads may each be provided as a dry reagent, or may be provided suspended in a solution. In some embodiments, the magnetic beads may be suspended in the nucleic acid precipitation reagent. In some embodiments the magnetic beads may be suspended in a different liquid phase, and the nucleic acid precipitation reagents may be mixed with the magnetic beads and the sample as part of the method 200. In some embodiments, the kit may also include wash and elution buffers. In some embodiments, the various reagents included in the kit (e.g., the first and second population of magnetic beads, the first and second nucleic acid precipitation buffers, the wash buffer, and/or the elution buffer) may generally include components and chemistries similar to those discussed with regards to the method 200 of FIG. 2.

In some embodiments, the kit may have components which are designed for a particular first size cutoff value (and in some embodiments a second size cutoff value). In some embodiments, the kit may include instructions which allow a user to tune a size cutoff (or both size cutoffs). For example, the kit may include a nucleic acid precipitation buffer, and instructions on which ratios of nucleic acid precipitation buffer to sample lead to which size cutoffs. The user may then determine which ratio/size cutoff to use in a particular application.

One example application for the method 200 of FIG. 2 may be the enrichment of fetal DNA from maternal DNA. On average, fetal cfDNA may generally be shorter than maternal cfDNA. Analysis of cfDNA may generally include sample collection (e.g., a blood draw), isolation of the cfDNA from the sample, preparation of a library from the isolated cfDNA. The library preparation may include adding DNA tags (e.g., ‘barcodes’) to one or both terminals of the cfDNA. The library preparation may generally elongate the cfDNA, for example, by 120 bp. Analysis of the cfDNA may also include next generation sequencing (NGS) and analysis of the data from the NGS.

In some embodiments, fetal DNA may be enriched by size selective filtering (e.g., method blocks 205 to 240) of the cfDNA after the cfDNA has been isolated from the sample, but before preparation of the library. The cutoff between fetal cfDNA and maternal cfDNA may generally be referred to as ‘X’. The value X may be used as the cutoff between short and long DNA for the method 200. In some embodiments, X may be 150 bp (e.g., normal maternal cfDNA may have a peak at 165 bp, and fetal DNA may be below 150 bp). Other size cutoff's (e.g. values of X) may be used in other example embodiments.

In some embodiments, the size selective enrichment of the DNA may be performed during the library preparation. The preparation of the library may generally include end repair and purification of the cfDNA, ligation of the cfDNA, which may include adding a DNA tag of a length which may be referred to as ‘Y’ (e.g., 120 bp), purification of the ligated cfDNA, gene panel enrichment, whole exome enrichment, amplification of the DNA (e.g., with PCR), and purification of the amplified DNA.

In some embodiments, the size selective enrichment (e.g., blocks 205-240) may be performed after the cfDNA end repair, and the first size cutoff may be X. In some embodiments, the size selective enrichment may be performed after the ligation and the size cutoff may be X+Y. In some embodiments, the size selective enrichment may be performed after the enrichment and X+Y may be used as the cutoff. In some embodiments, size selective enrichment may be performed after the amplification, and X+Y may be used as the cutoff. While the enrichment of fetal DNA has generally be described using magnetic beads as the substrate, the other substrates discussed in regards to the method 100 of FIG. 1 may also be used (e.g., spin column, electrophoresis, etc.).

In some embodiments, a workflow similar to that described above for fetal enrichment may be used for enrichment of tumor DNA. Enrichment of tumor DNA may also include blocks 245-260, which may allow for a comparison of the long DNA (e.g., the DNA above X, or above X+Y) to the short DNA.

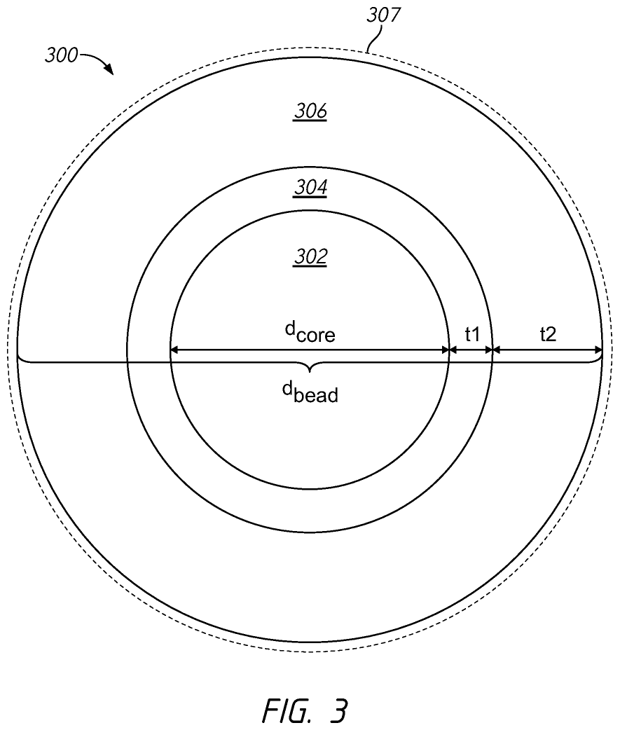

FIG. 3 is a cross-sectional diagram of a core-shell-shell magnetic bead according to some embodiments of the present disclosure. The core-shell-shell magnetic bead (CSS-MB) 300 includes a magnetic core 302, a first shell 304 and an outer shell 306. A population of the CSS-MBs 300 may, in some embodiments, be used as the first substrate and/or the second substrate of FIG. 1. For example, the first population of magnetic beads from the method 200 of FIG. 2 may include CSS-MBs 300, and the second population of magnetic beads from the method 200 of FIG. 2 may include CSS-MBs 300. In some embodiments, the first and the second population of magnetic beads may be identical except for their surface chemistry.

In some embodiments, the CSS-MB 300 may have a generally spherical shape, with a size defined by a diameter d_{bead}. The CSS-MBs 300 may have a diameter d_{bead} which may be 1 μm or less. For example, the diameter d_{bead} of a CSS-MB 300 may be between 200 nm and 1000 nm. Larger and smaller diameters may be used in other example embodiments. It should be understood that while CSS-MBs 300 may generally be shown as spherical for ease of discussion, this may represent an idealized view of a CSS-MB 300, and the CSS-MBs 300 may deviate from perfectly spherical. For example, a population of CSS-MBs 300 may include individual CSS-MBs 300 which are spherical, oblate, prolate, ellipsoid, have one or more other deviations from perfectly spherical (e.g., concave or convex ‘bumps’ on the surface), or combinations thereof.

The magnetic core 302 may have a diameter d_{core} of between 100 nm and 300 nm. The magnetic core 302 may be a solid material. In some embodiments the magnetic core 302 may be a solid cluster of crystals such as a cluster of superparamagnetic crystals. In some embodiments, the magnetic core 302 may be a single superparamagnetic crystal. In some embodiments the magnetic core may be formed of a metallic alloy such as a metal alloy oxide crystals. In some embodiments, the magnetic core 302 may include a mixture of metals at a selected ratio. For example, the magnetic core 302 may include a mixture of metals such as Fe, Co, and Ni. In some embodiments, the magnetic core 302 may generally have the composition Feₓ—Co_{y}—NiO_{z}, where x, y, and z are all integer values.

The first shell 304 may generally be a spherical layer deposited on an outer surface of the magnetic core 302. The first shell 304 may have a thickness t1, which in some embodiments may be between 10 nm to 50 nm. The first shell 304 may be a protective shell which prevents chemical interactions between the components of an environment outside the CSS-MB 300 and the magnetic core 302. The first shell 304 may be non-porous. The first shell 304 may be formed from an inert material. For example, the first shell 304 may include graphitic carbon. In some embodiments, the first shell 304 may have an outer surface which includes one or more chemical groups which promote bonding between the first shell 304 and the second shell 306. For example, in some embodiments where the first shell 304 includes graphitic carbon, the surface of the first shell 304 may include relatively abundant hydroxyl and/or epoxy groups which may provide anchoring sites for the second shell 306.

The second shell 306 may generally be a spherical layer deposited on an outer surface of the first shell 304. The second shell 306 may have a thickness t2, which in some embodiments may be between 20 nm to 100 min. Accordingly, the CSS-MB 300 may have an overall radius that is the radius of the magnetic core 302 plus the thickness t1 and thickness t2 of the first shell 304 and second shell 306 respectively.

The second shell 306 may be an outer shell of the CSS-MP 300. The second shell 306 may further protect the magnetic core 302 (e.g., in addition to the protection offered by the first shell 304), may provide surface chemistry useful for one or more reactions, and/or provide active sites which may be functionalized with additional chemical groups to provide such surface chemistry. In some embodiments, the second shell 306 may be non-porous. In some embodiments, the second shell 306 may be porous. For example, the second shell 306 may include mesopores which may evenly distribute throughout the second shell 306. In some embodiments the second shell may be formed from silicon oxide, such as a condensed silicon oxide.

In some embodiments, an outer surface the second shell 306 may be surrounded by a layer of chemical groups 307. These chemical groups 307 may be inherent to the chemistry of the second shell 306 and/or may be due to modification of the surface chemistry of the second shell. For example, the chemical groups 307 may include chemicals which are bound to the outer surface of the second shell 306. The surface chemical groups 307 may be used to improve the chemical properties of the CSS-MB 300 in a solution (e.g., their dispersal in solution) and/or to promote the binding between the CSS-MB 300 and one or more biomolecules such as nucleic acids.

In some embodiments, various functional groups may be bound to the surface of the CSS-MB as part of the chemical groups 307. The second shell 306 may include and/or may be modified to include a number of active sites which may be used to bind the functional groups to the outer surface of the second shell 306. For example, in embodiments where the second shell 306 is formed of silicon dioxide, the second shell 306 may include a number of surface hydroxyl groups. These surface hydroxyl groups may be modified to into carboxylic groups, which may form active sites for the binding of one or more functional groups.

One example of chemical groups 307 may include chemicals to promote binding between biomolecules. For example, the surface hydroxyl groups of the CSS-MB 300 may be modified with chemicals presenting carboxylic groups such as small molecules, polymers, dendrimers containing an epitope of carboxylic groups, by covalently binding these chemicals to the hydroxyl groups. For example, the hydroxyl groups may be converted to carboxylic groups with chemicals such as succinic anhydride, polyacrylic acid, amino acids, alginic acid, carbonic acid, malic acid, tartaric acid, citric acid, salicylic acid, gallic acid, sialic acid, and combinations thereof. The carboxylic groups may be useful to enable binding of certain target biomolecules based on the mechanism of solid-phase reverse immobilization under certain buffer conditions.

The chemical groups 307 may also include hydrophilic components (e.g., hydrophilic moieties) which may alter the overall surface chemistry of the CSS-MB 300. For example, the hydrophilic components may be small molecules (e.g. zwitterionic molecule, sugar molecule) or polymers (e.g. polyethylene glycol, poly vinyl alcohol, polyethyleneimine). The hydrophilic components may increase the dispersibility of magnetic beads in aqueous solutions, which may be beneficial for interaction between targeted biomolecules and the CSS-MBs 300. The hydrophilic components in the chemical groups 307 may also help avoid the non-specific binding of other biomolecules in the samples, which in turn may enhance the purity of enriched targeted biomolecules. For example, hydrophilic components may include polyethylene glycol, polyacrylic acid, polyvinyl alcohol, glucose, sucrose, cysteine, maltose, chitosan, alginate, cellulose, chitin, starch and combinations thereof.

### Example 1—Removal and Recovery of DNA Fragments of Different Size

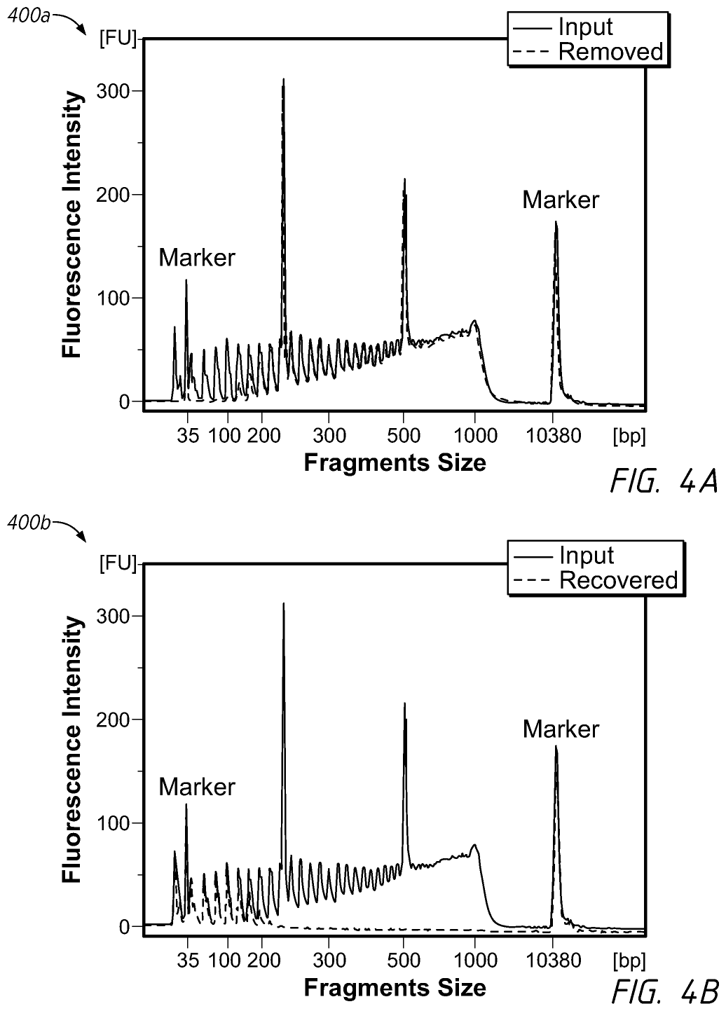

FIGS. 4A-4B are graphs depicting the separation of nucleic acids by size according to some embodiments of the present disclosure. The graphs 400a of FIG. 4A and 400b of FIG. 4B show DNA removed from a population of DNA and DNA remaining after the DNA is removed respectively. The graphs 400a-b show DNA fragment size (in bp) along a horizontal axis, and concentration of DNA at that size (measured by fluorescence intensity) along the vertical axis. Both graphs 400a-b show a first trace which is the ‘input’, a set of DNA fragments. The graph 400a shows a trace of ‘removed’ DNA which may represent DNA above a cutoff size (e.g., the long DNA fragments of FIG. 2). The graph 400b shows a trace of ‘recovered’ DNA, which may represent DNA below a cutoff size (e.g., the short DNA fragments of FIG. 2).

An input of 20-1000 bp DNA fragments is used as an example to demonstrate the removal of long fragments and recovery of short fragments using the method 200 of FIG. 2. The input DNA was processed as: 1) mixing with one type of magnetic beads (a first population of magnetic beads); 2) separating the first population of magnetic beads from the remaining solution; 3) mixing the supernatant with a different kind of magnetic beads (a second population of magnetic beads); 4) Washing the separated first and second population of magnetic beads in each container with 70% ethanol solution; 5) Eluting the DNA from the each population of magnetic beads with elution buffer. The eluates are compared with the input using a commercial system, such as the Agilent Bioanalyzer 2100. The eluate from the first population of magnetic beads contain long fragments and the signal overlaps with the input in the range of 200-1000 bp, indicating the long fragments are removed from the input during the workflow. The eluate from the second population of magnetic beads contain short fragments and the signal generally overlaps the input DNA fragments in the range of 20-200 bp, showing the efficient recovery of small fragments, as short as 20 bp. The overlapping signals of the eluates and the input in the range of 200-1000 bp as well as 20-200 bp demonstrates the workflow of preferential selection of short fragments from DNA of various sizes.

### Example 2—Enrichment of Small DNA Fragments from a cfDNA Standard

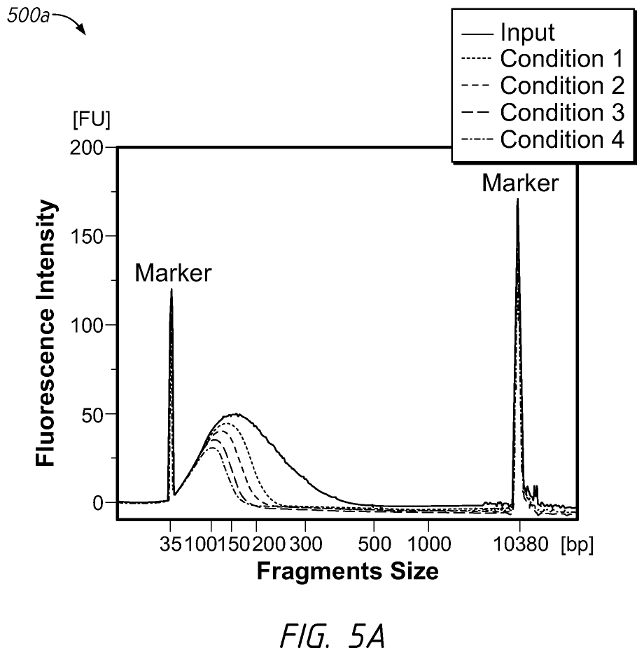

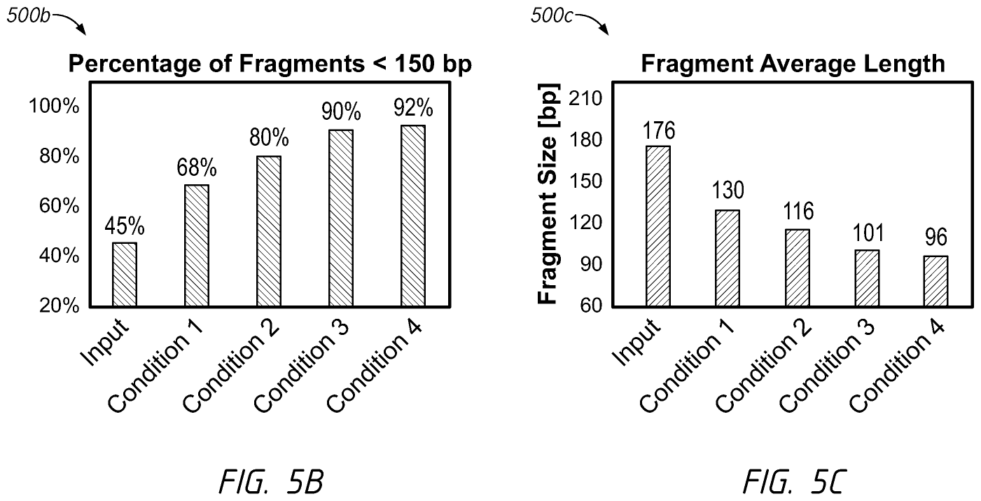

FIGS. 5A-5C are graphs depicting enrichment of small DNA fragments from a cell-free DNA (cfDNA) sample according to some embodiments of the present disclosure. The experiments of Example 2 show an example of how the cutoff size may be tuned by varying the ratio of nucleic acid precipitation buffer to sample. FIG. 5A shows a graph 500a which shows DNA fragment size across the horizontal axis and concentration along the vertical (similar to the graphs 400a-b of FIGS. 4A-B). The graph 500a shows different traces each of which represents an input and outputs after using various nucleic acid precipitation buffer conditions. FIGS. 5B-C show bar graphs 500b-c respectively, each of which show different bars for the input and different conditions. The vertical axis of FIG. 500b shows a percentage of DNA fragments below 150 bp in the recovered DNA fragments or in the input. The vertical axis of FIG. 500c shows the average fragment size in the recovered DNA fragments compared with the input.

A commercial cfDNA standard (35-500 bp) sample is processed to enrich the short DNA fragments (e.g., with the method 200 of FIG. 2). Four nucleic acids precipitation reagent conditions are used to enrich DNA fragments less than 150 bp by adjusting the reagent volume to sample volume ratio (1.2×, 10.4×, 1.6×, and 1.8×) to achieve different enrichment effects. In general, the process includes: 1) mixing input cfDNA standards with a first type of magnetic beads and 1.2, 1.4, 1.6, and 1.8 times volume of corresponding nucleic acids precipitation reagent respectively, to selectively bind long DNA to the first type of magnetic beads with a tuned cutoff value based on the volume ratio; 2) separating the magnetic beads from the solution under external magnetic field; 3) collecting the supernatant solution which includes the unbound short DNA fragments; 4) mixing the supernatant with a second type of magnetic bead, at least one type of alcohol, and nucleic acids precipitation reagents to bind the remaining short DNA; 5) separating the second type of magnetic beads from the solution under external magnetic field and discarding the supernatant; 6) washing the magnetic beads with 70% ethanol solution; 7) eluting the selected DNA from the second type of magnetic beads with an elution buffer. The enriched DNA is characterized and the fragments fraction and average size are calculated, for example using a commercial instrument and software such as the Agilent 2100 bioanalyzer software. Comparing to the original input, the four enriched samples increased the fraction of DNA <150 bp from 45% (original input) to 68%, 80%, 90%, and 92%, respectively. The four enriched samples have decreased average length from 176 bp (original input) to 130 bp, 116 bp, 101 bp, and 96 bp respectively. Accordingly, the amount of short (e.g., below the cutoff) DNA is enriched compared to the input.

### Example 3—Enrichment of Small DNA Fragments from Human cfDNA Sample

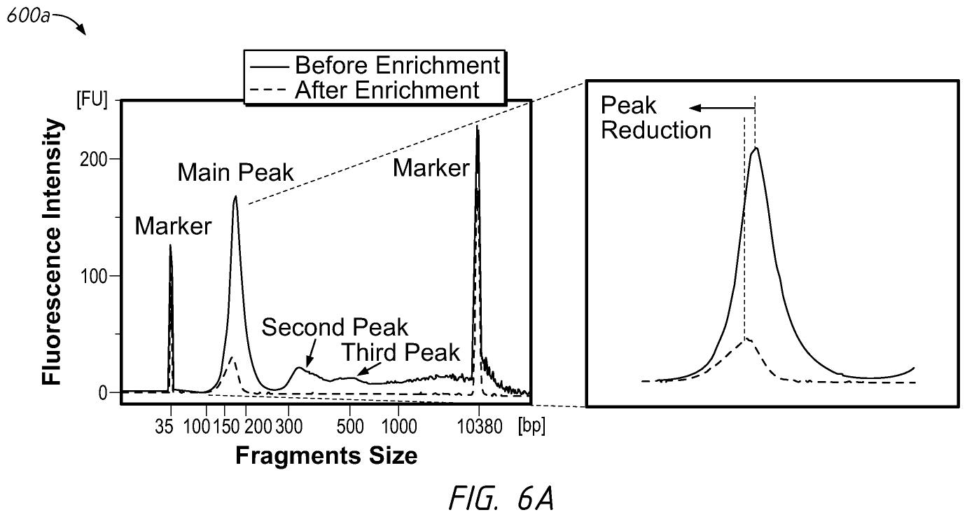

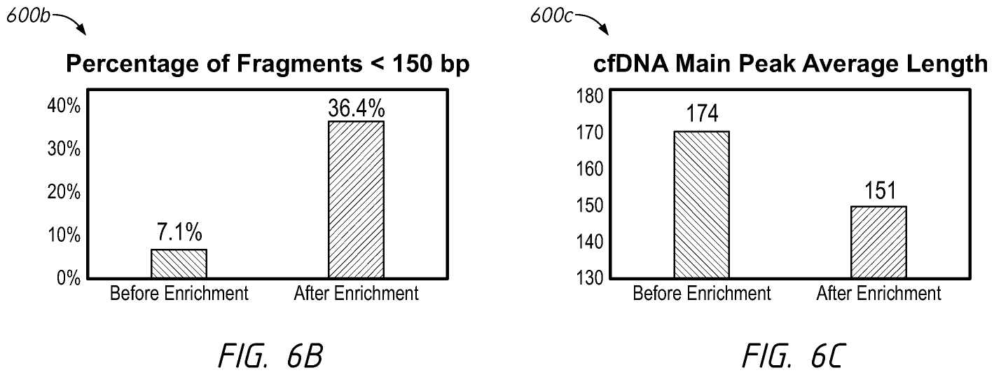

FIGS. 6A-6C are graphs of enrichment of small DNA fragments from human cell-free DNA (cfDNA) sample according to some embodiments of the present disclosure. FIG. 6A shows a graph 600a of DNA fragment size along the horizontal axis and concentration along the vertical axis (e.g., similar to the graph 400a-b of FIGS. 4A-4B and 500a of FIG. 5A). The graph 600a shows a first trace which is the DNA fragments before enrichment, and a second trace which is after enrichment (e.g., enrichment with the method 200 of FIG. 2). The graph 600a includes an inset which shows a main peak of the DNA fragments. The graphs 600b and 600c are similar to the graphs 500b and 500c of FIGS. 59-5C and show percentage of DNA fragments <150 bp and average length of DNA fragments in the main peak, respectively.

Fetal-specific cffDNA may have relatively short fragment size comparing to maternal cfDNA. Accordingly, enrichment of the short fragments (e.g., less than 150 bp) may increase the fetal fraction (e.g., the percentage of DNA in the sample from the fetus). A total of 21 maternal plasma from pregnant women with male fetus are collected with informed consent and the cfDNA is isolated to be processed for enrichment of the short fragments (e.g., less than 150 bp) in the sample. The example experiments of FIGS. 6A-6C may include an enrichment process which is similar to the methods 100 of FIG. 1 and/or 200 of FIG. 2). In brief, the enrichment process may include: 1) mixing input cfDNA sample with a first type of magnetic beads and a corresponding first nucleic acids precipitation reagent to selectively bind long DNA to the first type of magnetic beads; 2) separating the magnetic beads from the solution under external magnetic field; 3) collecting the supernatant solution containing unbound short DNA fragments; 4) mixing the supernatant with a second type of magnetic beads and a second nucleic acid precipitation reagent including an alcohol to bind the remaining short DNA; 5) separating the second type of magnetic beads from the solution under external magnetic field and discarding the supernatant; 6) washing the magnetic beads with 70% ethanol solution; 7) eluting the selected DNA from the second type of magnetic beads with an elution buffer.

As an example, cfDNA before and after the enrichment in case 0 is characterized using a commercial instrument such as the Agilent Bioanalyzer 2100. Comparing to the original cfDNA input, DNA fragments <150 bp are enriched. The method also removes genomic DNA contamination as well as the second and third peaks of cfDNA at 350 and 525 bp, which are mostly maternal genomic materials and undesired for NIPT. Quantitative analyses of enriched samples indicate the increased fraction of DNA <150 bp from 7.1% to 36.4% in the enriched sample, and the average length of the cfDNA main peak decreased from 174 bp (original input) to 151 bp. The enrichment of short DNA fraction is shown by the decreased average length as well as the removal of genomic DNA contamination.

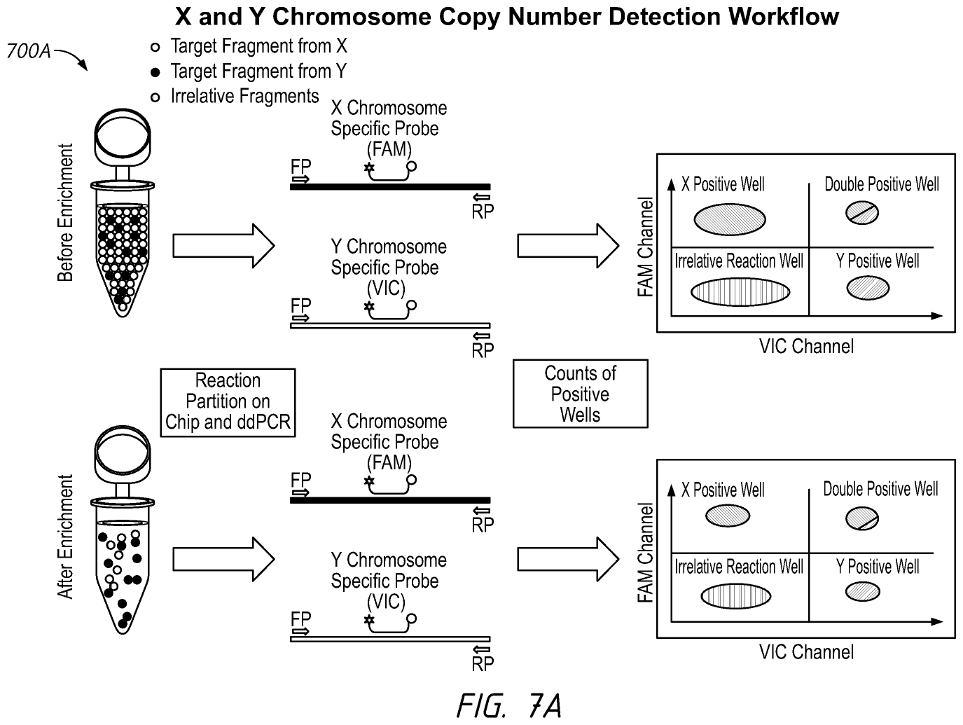

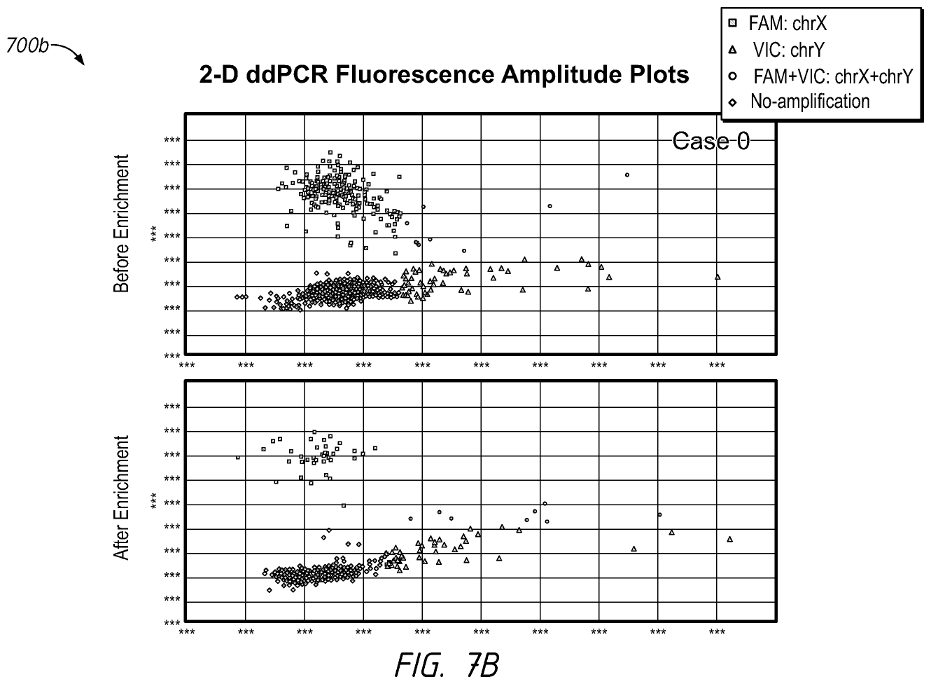

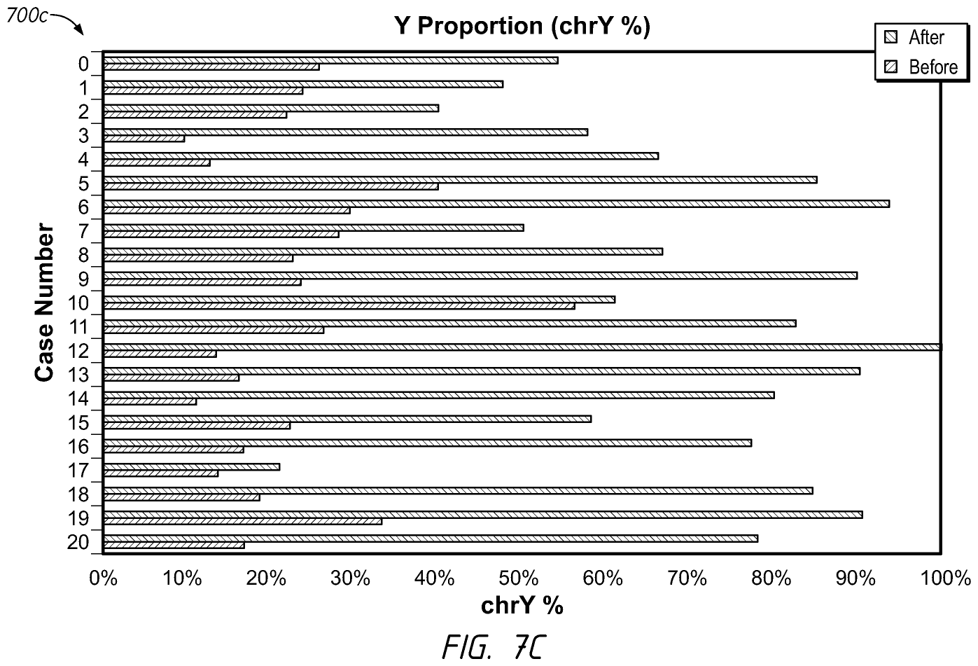

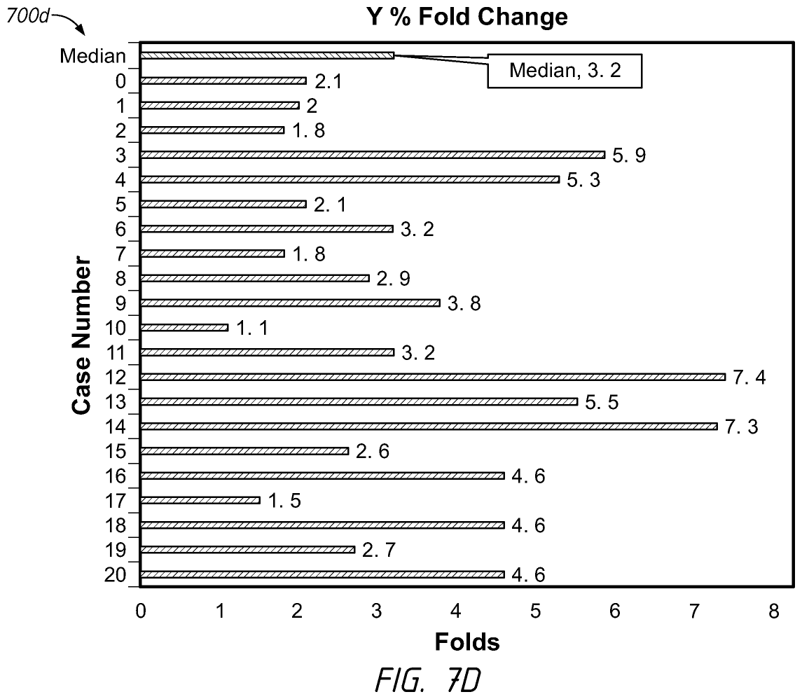

FIGS. 7A-7D are graphs showing the enrichment of a fetal fraction of DNA assessed by the quantification of the Y chromosome according to some embodiments of the present disclosure. The enrichment may be done using a method similar to the method 100 of FIG. 1 and/or 200 of FIG. 2. Enrichment of fetal fraction is performed on cfDNA isolated from the blood drawn from mothers carrying male fetuses. Accordingly, only the fetal DNA may include Y chromosomes, and thus the Y chromosome may be used as a proxy for fetal DNA. FIG. 7A shows a graph 700a which is a schematic representation of the characterization of fetal fraction enrichment. FIG. 7B shows a graph 700b which shows the population of X and Y and chromosomes before and after enrichment. FIG. 7C shows a graph 700c of the proportion of Y chromosomes. FIG. 7D shows a graph 7D of a percentage change in the concentration of Y chromosomes.

The fetal fraction is assessed by the quantification of Y chromosome. The copy number of X and Y chromosome is quantified using a ddPCR method. The probe for the ddPCR is designed to target the XIST (Xq13) gene on the X chromosome, or the SRY (Yp11) gene on the Y chromosome. XIST and SRY are located in conserved regions of the X and Y chromosome, respectively. As shown in a representative 2-D ddPCR fluorescence amplitude plot of samples before and after the enrichment workflow (e.g., graph 700b), the positive wells of chrX (FAM fluorescent tags), chrY (VIC fluorescent tags), chrX+chrY (FAM+VIC fluorescent tags), and wells of no-amplification and undetermined are used to calculate the copy number of chrX and chrY using a ProFlex™ 2× Flat PCR System. The number of chrX positive wells are significantly reduced after the enrichment workflow while chrY positive wells remain at a similar level, suggesting the increased Y chromosome fraction after the enrichment workflow. The Y proportion (e.g., Y %) may be calculated using Equation 1, below:

Y%=copy number of chrY/(copy number of chrX+copy number of chrY) Eqn. 1

The enrichment is performed on 21 maternal samples. The results of the maternal samples both before and after enrichment, are summarized in Table 1, below:

TABLE 1
Summary of Maternal Samples

| | | | | Before Enrichment | | | After Enrichment | | |
|---|---|---|---|---|---|---|---|---|---|
| Case | | | | chrX, | chrY, | chrY | chrX, | chrY, | chrY |
| Number | Gestation | | Fetus | copies/μL | copies/μL | % | copies/μL | copies/μL | % |
| 0 | 31 2/7 | weeks | Male | 214 | 74.5 | 25.8% | 37.6 | 45.1 | 54.5% |
| 1 | 38 1/7 | weeks | Male | 67.4 | 21.1 | 23.8% | 8.4 | 7.6 | 47.5% |
| 2 | 16 2/7 | weeks | Male | 86.6 | 24.1 | 21.8% | 4.8 | 3.2 | 40.0% |
| 3 | 17 2/7 | weeks | Male | 48.6 | 5.3 | 9.8% | 2.1 | 2.9 | 58.0% |
| 4 | 16 | weeks | Male | 34.6 | 5 | 12.6% | 2.9 | 5.7 | 66.3% |
| 5 | 16 | weeks | Male | 29.7 | 19.8 | 40.0% | 3.7 | 21.2 | 85.1% |
| 6 | 18 5/7 | weeks | Male | 41 | 17.1 | 29.4% | 0.7 | 10.6 | 93.8% |
| 7 | 17 5/7 | weeks | Male | 53.4 | 21 | 28.2% | 4.3 | 4.3 | 50.0% |
| 8 | 16 5/7 | weeks | Male | 19.3 | 5.7 | 22.8% | 2.9 | 5.8 | 66.7% |
| 9 | 24 4/7 | weeks | Male | 39.4 | 12.2 | 23.6% | 0.7 | 6.4 | 90.1% |
| 10 | 16 4/7 | weeks | Male | 17.5 | 22.6 | 56.4% | 5.1 | 8 | 61.1% |
| 11 | 19 4/7 | weeks | Male | 32.9 | 11.7 | 26.2% | 2.9 | 14 | 82.8% |
| 12 | 18 1/7 | weeks | Male | 27.5 | 4.3 | 13.5% | 0 | 8.6 | 100.0% |
| 13 | 30 6/7 | weeks | Male | 28.7 | 5.6 | 16.3% | 2.3 | 21.7 | 90.4% |
| 14 | 20 5/7 | weeks | Male | 34.9 | 4.3 | 11.0% | 0.7 | 2.8 | 80.0% |
| 15 | 16 3/7 | weeks | Male | 20.3 | 5.8 | 22.2% | 7.5 | 10.4 | 58.1% |
| 16 | 31 4/7 | weeks | Male | 35.4 | 7.2 | 16.9% | 3.6 | 12.3 | 77.4% |
| 17 | 18 6/7 | weeks | Male | 44.6 | 7.1 | 13.7% | 8.16 | 2.2 | 21.2% |
| 18 | 33 1/7 | weeks | Male | 34.6 | 7.9 | 18.6% | 1.5 | 8.3 | 84.7% |
| 19 | 17 6/7 | weeks | Male | 21.9 | 10.9 | 33.2% | 0.8 | 7.6 | 90.5% |
| 20 | 23 6/7 | weeks | Male | 38.4 | 7.8 | 16.9% | 1.4 | 5 | 78.1% |

The 21 samples tested (e.g., as shown in Table 1) all have enrichment effect after the workflow, although the fold change of Y % varies among different samples. The fold change of Y proportion before and after the enrichment is from 1.1 to 7.4 and the median is 3.2. By preferentially enriching the short fragments of cfDNA samples, the Y % can be significantly increased, indicating fetal fraction enrichment which may lead to potential improvement of sensitivity and specificity in NWT applications.

Accordingly, by using a first and second substrate, nucleic acids below a cutoff size may be recovered and may be washed to remove impurities and/or enriched to increase a concentration of the target biomolecule. The use of two different substrates may also increase the efficiency of the filtering as well as allowing for additional size selective filtration, for example to retain nucleic acids between a first and second size threshold. The use of two different substrates may also allow the filtered nucleic acids (e.g., the long nucleic acids) to be recovered, which may be useful in applications involving a comparison of the ratio of nucleic acids above and below the cutoff.

It is to be appreciated that any one of the examples, embodiments or processes described herein may be combined with one or more other examples, embodiments and/or processes or be separated and/or performed amongst separate devices or device portions in accordance with the present systems, devices and methods.

It is to be appreciated that while certain numerical values have been given, these are for example only. A numerical value given as such an example should be considered approximate, and values which are greater or lesser than the example numerical value may also be used. Similarly, when a range of numerical values (e.g., between X and Y) is given, it should be understood to be inclusive of the boundaries (e.g., X and Y).

It is to be appreciated that any of the methods of the present disclosure may include additional steps, that certain steps may be omitted, and that the steps may be performed in any order. For example, a method which describes a first step as being generally followed by a second step may include additional steps between the first and second steps. In some embodiments, the second step may precede the first step.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

What is claimed is:

1. A method comprising:
mixing a sample including nucleic acids with a first substrate;
separating nucleic acids above a first size threshold from a remainder of the sample with the first substrate;
mixing the nucleic acids in the remainder of the sample with a second substrate; and
recovering nucleic acids below the first size threshold from the second substrate.

2. The method of claim 1, further comprising recovering the nucleic acids above the first size threshold.

3. The method of claim 2, further comprising binding the nucleic acids above the first size threshold to a first substrate comprising magnetic beads, and wherein recovering the nucleic acids above the first size threshold includes eluting the nucleic acids above the first size threshold from the magnetic beads.

4. The method of claim 1, wherein the first substrate comprises a first population of magnetic beads, and wherein the second substrate comprises a second population of magnetic beads.

5. The method of claim 4, wherein the first population of magnetic beads and the second population of magnetic beads include core-shell-shell magnetic beads.

6. The method of claim 4, wherein separating the bound nucleic acids above the first size threshold includes applying a magnetic field to the sample and removing a supernatant comprising the remainder of the sample.

7. The method of claim 4, wherein the first population of magnetic beads includes surface chemistry of carboxylic groups configured to selectively bind to the nucleic acids above the first size threshold.

8. The method of claim 4, wherein the second population of magnetic beads includes surface chemistry of hydroxyl groups.

9. The method of claim 8, further comprising binding the nucleic acids in the remainder of the sample to the second population of magnetic beads by mixing the remainder of the sample with a nucleic acid precipitation reagent including at least one alcohol.

10. The method of claim 1, further comprising separating nucleic acids above a second size threshold from the remainder of the sample with the second substrate, wherein the second size threshold is smaller than the first size threshold, and wherein the recovered nucleic acids are above the second size threshold and below the first size threshold.

11. The method of claim 1, further comprising binding the nucleic acids above the first size threshold to the first substrate in the presence of a first nucleic acid precipitation reagent; and binding the nucleic acids in the remainder of the sample to the second substrate in the presence of a second nucleic acid precipitation reagent.

12. The method of claim 11, wherein at least one of the first nucleic acid precipitation reagent and the second nucleic acid precipitation reagent comprise dehydrating agents, salt bridges, buffering agents, carrier molecules, surfactant, and combinations thereof.

13. The method of claim 1, further comprising washing the second substrate with a wash buffer.

14. The method of claim 1, wherein recovering the nucleic acids below the first size threshold from the second substrate includes eluting the nucleic acids from the second substrate with an elution buffer.

15. The method of claim 1, wherein the nucleic acid comprises DNA, RNA, oligos, nucleic acids labeled with radioactive phosphates, fluorophores, nucleotides modified with biotin or digoxygenin, or combinations thereof.

16. The method of claim 1, wherein the first substrate comprises magnetic beads, non-magnetic beads, a gel, a capillary tube, or a spin column and wherein the second substrate comprises magnetic beads, non-magnetic beads, a gel, a capillary tube, or a spin column.

17. The method of claim 1, wherein separating the nucleic acids above the first size threshold comprises applying a magnetic field, applying an acceleration, centrifuging the sample, or applying a potential to the first substrate.

18. A kit comprising:
a first substrate;
a first nucleic acid precipitation reagent;
a second substrate; and
a second nucleic acid precipitation reagent,
wherein the first substrate is configured to bind nucleic acids above a first size threshold in the presence of the first nucleic acid precipitation reagent, and wherein the second substrate is configured to bind nucleic acids in the present of the second nucleic acid precipitation reagent.

19. The kit of claim 18, wherein the first substrate is a first population of magnetic beads, and wherein the second substrate is a second population of magnetic beads.

20. The kit of claim 19, wherein the first population of magnetic beads and the second population of magnetic beads include core-shell-shell magnetic beads.

21. The kit of claim 18, wherein the second substrate is configured to bind nucleic acids above a second size threshold in the presence of the second nucleic acid precipitation reagent.

22. The kit of claim 18, wherein at least one of the first nucleic acid precipitation reagent or the second nucleic acid precipitation reagent comprise dehydrating agents, salt bridges, buffering agents, carrier molecules, surfactant, and combinations thereof.

23. The kit of claim 22, where the dehydrating agents comprise polyalkylene glycols with a molecular weight between 1000 to 10,000, and a weight to volume concentration between 10% to 25%.

24. The kit of claim 22, wherein the salt bridge comprises NaCl, KCl, CaCl₂ or combinations thereof, with a concentration from 0.5 M to 5 M.

25. The kit of claim 22, wherein the buffering agents comprise Tris with a concentration of 0-10 mM, EDTA with a concentration of 0-1 mM, Tris-HCl with a concentration of 0-10 mM, and combinations thereof.

26. The kit of claim 22, wherein the carrier molecules comprise Sodium Acetate with a concentration of 0.3-1M, Lithium Chloride with a concentration of 0.1-1M, Glycogen with a concentration of 0.1-2 uM, Ammonium acetate with a concentration of 0.5-2M, Linear Polyacrylamide with a concentration of 10-20 ug/mL, and combinations thereof.

27. The kit of claim 22, wherein the surfactant comprises Tween-20 with a concentration of 0.01%-0.5%, Triton X-100 with a concentration of 0.01%-0.5%, SDS with a concentration of 0.1%-1%, and combinations thereof.

28. The kit of claim 18, further comprising an alcohol comprising ethanol with a concentration of 45-85%, isopropanol with a concentration of 33%-70%, and combinations thereof.

29. The kit of claim 18, further comprising a washing buffer comprising at least one alcohol.

30. The kit of claim 18, further comprising an elution buffer comprising Tris with a concentration of 0-10 mM, EDTA with a concentration of 0-1 mM, Tris-HCl with a concentration of 0-10 mM, and combinations thereof.

31. A method comprising:
collecting a sample including nucleic acids;
isolating the nucleic acids from the sample;
filtering the nucleic acids to retain selected ones of the nucleic acids below a size threshold using a first substrate configured to selectively separate nucleic acids above the size threshold from the selected ones of the nucleic acids and a second substrate configured to separate the selected ones of the nucleic acids from impurities;
preparing a library based on the isolated nucleic acids; and
sequencing the prepared library.

32. The method of claim 31, wherein filtering occurs after isolating the nucleic acids and before preparing the library.

33. The method of claim 31, wherein preparing the library comprises elongating the isolated nucleic acids.

34. The method of claim 33, wherein filtering the nucleic acids occurs after the isolated nucleic acids are elongated, and wherein the size threshold is based on a target length of nucleic acids and an amount of elongation.

35. The method of claim 31, wherein the nucleic acids are cell free DNA (cfDNA), DNA, RNA, oligos, labelled nucleic acids, modified nucleic acids, or combinations thereof.

36. The method of claim 31, wherein the nucleic acids below the size threshold comprise fetal DNA.

37. The method of claim 31, wherein the nucleic acids below the size threshold comprise tumor DNA.

38. The method of claim 31, further comprising recovering the nucleic acids above the size threshold.

39. The method of claim 31, wherein filtering the nucleic acids comprises:
binding the non-selected ones of the nucleic acids to the first substrate; and
applying an external force to separate the first substrate and the bound non-selected ones of the nucleic acids from unbound ones of the nucleic acids.

40. The method of claim 39, wherein the first substrate comprises magnetic beads and wherein the external force includes a magnetic field.

41. The method of claim 39, wherein the filtering further comprises:
binding the selected ones of the nucleic acids to the second substrate;
applying an external force to separate the second substrate and the bound selected ones of the nucleic acids from the impurities; and
recovering the selected ones of the nucleic acids from the second substrate.
